(19) United States
(12) Patent Application Publication
(10) Pub. No.: US 2013/0274542 A1
(43) Pub. Date: Oct. 17, 2013

(54) MEDICAL DEVICE FOR TREATMENT OF HUMAN PARASITISM AND OF RELATED DISEASES

(71) Applicants: Cataldo Volo, Mesenzana (VA) (IT); Gianmarco Malcotti, Porto Valtravaglia (VA) (IT); Domenico Toneatto, Reinach AG (CH)

(72) Inventors: Cataldo Volo, Mesenzana (VA) (IT); Gianmarco Malcotti, Porto Valtravaglia (VA) (IT); Domenico Toneatto, Reinach AG (CH)

(73) Assignee: PERISO SA, Isone (CH)

(21) Appl. No.: 13/641,740

(22) PCT Filed: Apr. 30, 2010
(86) PCT No.: PCT/IB2010/051907

§ 371 Date: Jul. 1, 2013

(51) Int. Cl. A61N 2/00

(52) U.S. Cl. CPC A61N 2/004
USPC 600/13

(57) ABSTRACT

The use of a magnetic field for eliminating the malaria parasite is disclosed. Furthermore, herein described is an electromedical device for the treatment of human parasitosis, and especially the malaria parasite, comprising a plurality of coils (10) defining a cage (S) for accommodating the patient, the coils being connected to a drive circuit for subjecting the patient to a substantially uniform magnetic field.

### FIELD OF THE INVENTION

The invention describes an electromedical device for the treatment of human parasitism, and of related diseases. The invention in particular regards the reduction of the malaria parasite and treatment of malaria.

### PRIOR ART

As known, malaria is a parasitosis currently representing one of the most common infectious diseases worldwide. Up to date, malaria is an endemic disease in many tropical and subtropical areas, and affects millions of people each year. The disease is caused by a parasite whose main vector is represented by the anopheles mosquito. In particular, it is known that the parasites responsible for malaria are plasmodium protozoa.

The therapy, according to the prior art, is substantially based on a class of drugs known as antimalarial drugs. However, these drugs reveal drawbacks among which the occurrence of adverse reactions, and in addition they also cause the development of drug-resistant parasite strains, hence they may lose efficiency. These drawbacks are commonly observed in therapies based on drugs, and thus difficult to overcome. In particular the development of drug-resistant parasite strains leads to search for more potent drugs, which however tend to cause adverse effects that are more harmful to humans and/or the environment.

Analogous considerations may also apply, more generally, to other parasitosis and especially to the endemic ones.

### SUMMARY OF THE INVENTION

The applicant has observed that parasitosis, and in particular the malaria parasite, may be eliminated by exposing the person infected by such parasitosis to a magnetic field of suitable intensity and frequency.

It is assumed without being bound by the theory, that the magnetic field damages the defence of the parasite, and in particular its outer protection membrane, causing death of the parasite within a short period of time.

Preferably, the magnetic field has an intensity reaching up to 100 gauss (i.e. 10 millitesla mT) and frequency between 0.5 and 50 Hz. For example, there have been positive observations regarding the treatment of people suffering from malaria using a magnetic field of 4 gauss (0.4 mT) and 10 Hz.

The magnetic field is preferably generated with a plurality of coils, each coil being made up of a given number of turns. The coils are preferably powered by means of a power supplier which generates a sinusoidal, triangular or square wave signal. In particularly the waveform is preferably generated by means of an oscillator.

Thus, according to the invention, there is provided a device for the treatment of human parasitosis, and especially of the malaria parasite, comprising a plurality of coils which define a cage for accommodating at least one patient, the device comprising at least one drive circuit, the coils being connected to said circuit, said drive circuit and said coils being arranged to generate a substantially uniform magnetic field in a region inside the accommodation cage.

The coils are preferably wound on a non-magnetic material support, for example aluminium or PVC. According to a preferred embodiment, the coils are substantially coaxial windings and they are slidable on longitudinal supports.

Preferably, the coils are substantially formed by square coaxial windings and each coil is fixed on corner structures slidable in longitudinal direction. The sliding coils allow adjusting the distance between the coils to obtain the uniformity of the magnetic field. Other types of coils applicable to the invention comprise: cylindrical coil, Helmholtz coil or Maxwell coil.

Each of the coils has a winding with a suitable number of turns. In preferred applications, the voltage of the coils is about 50 V and the number of turns is lower than 150, for example between 10 and 150. The windings are connected in series or parallel. Preferred embodiments are obtained with a number of coils between four and six, but a higher or lower number may be used.

The drive circuit is preferably suitable to supply the coils with a voltage having a constant value. Preferably, said circuit supplies a continuous voltage on a number of channels equivalent to the number of coils. In preferred embodiments, the voltage to be supplied to the coils is about 50 Vdc and the resistance of the coils is between 3 and 12 ohm approximately; thus, the drive circuit may use a technology similar to the amplification technology which is known in the acoustic diffusion industry. The power required for practical applications, for exemplifying purposes, may amount to a few hundreds of watts per channel, for example 600 W/channel. A class D switching amplifier is preferably used. Such amplifier is preferred for the capacity of providing the power required with least thermal dissipation.

The drive circuit may comprise a voltage or current amplifier.

The supply signal provided at the input of said amplifier is for example generated by an oscillator and preferably selected between sinusoidal, triangular, or square wave. According to another aspect of the invention, the drive circuit comprises a synthesizer for generating the signal supplied to the amplifier; furthermore, the synthesizer also allows selecting the waveform from among a given number of predetermined waveforms.

The invention allows obtaining advantages in the treatment of people affected by the malaria parasitosis. The patient is accomodated in the space between the coils (accommodation area) and subjected—for a given period of time—to the magnetic field generated by the coils themselves. Substantially it has been observed a surprising efficiency of the biomagnetism phenomenon in treating parasitosis, and in particular the malaria parasitosis. The main advantage of biomagnetism with respect to the therapy using drugs, lies in the absence of adverse effects and thus applicability almost to any subject of any age. Furthermore, the parasite does not undergo mutations leading to new drug-resistant strains always more and more difficult to treat.

Thus, an aspect of the invention consists in using a magnetic field of suitable intensity and frequency for eliminating the malaria parasite. Preferably, the magnetic field has an intensity of up to 100 gauss and frequency between 2 and 50 Hz; even more preferably, it has an intensity of about 4 gauss and frequency of about 10 Hz. The treatment may be performed directly on a person suffering from malaria or for eliminating the parasite where present. For example, the invention may be usefully applied for reducing the malaria parasite in infected waters.

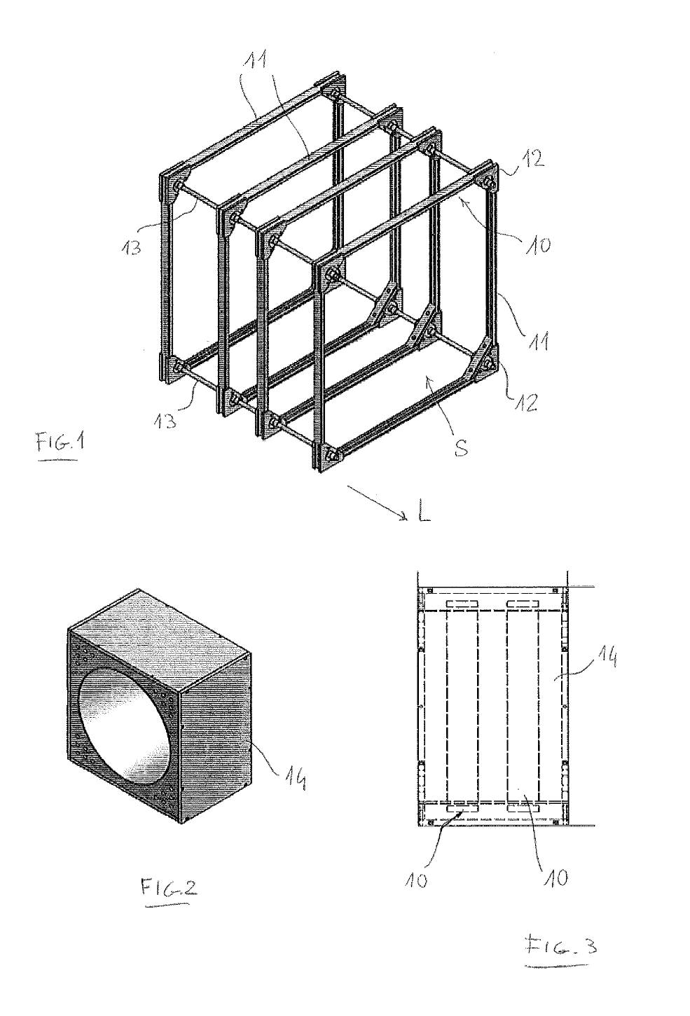

An exemplifying embodiment of the device according to the invention shall now be described with reference to the attached FIGS. 1 to 3, wherein:

FIG. 1 shows an exemplifying system made up of 4 coils,

FIG. 2 shows a system made up of two coils wound around a pipe made of non-magnetic material, in perspective view,

FIG. 3 is a side view of the system of FIG. 2.

FIG. 1 schematically shows a machine according to the invention, which develops to form a cage, extended in longitudinal direction L, and it is made up of a plurality of coils. Shown in the example are four coils 10; the number of coils varies depending on the embodiments, preferably between four and six. In the example, the coils are square-shaped and they substantially represent square coaxial windings, having a defined side. Other shapes, for example circular, may be provided for.

The coils are made up of rectilinear elements 11 and corner joints 12. The joints are preferably slidable on guides 13, in such a manner that the coils 10 may be moved away or drawn near to each other, by moving them towards direction L. For example, the side of the abovementioned coils 10 may be about 1.2 metres.

The coils 10 form a cage having an internal space S for accomodating the patient. For example, the patient may lie along direction L, in such a manner that during the process the body is exposed to the magnetic field generated by the same coils.

Referring to FIGS. 2 and 3, a treatment machine according to the invention is obtained, in another embodiment, by means of two coils 10 (indicated with the dashed line) wound around a shell or pipe 14 preferably made of PVC, for example said PVC pipe has a diameter of about 315 mm.

The machine comprises a drive circuit suitable to drive the coils with a substantially constant current. The drive circuit comprises a signal generator and an amplifier; said amplifier preferably being a multichannel amplifier with a channel for each of the coils 10, capable of supplying about 600 W per coil with a voltage of about 50 V.

1-12. (canceled)

13. An electromedical device for the treatment of human parasitosis, comprising:
a plurality of coils defining a cage for accommodating at least one patient; and
at least one drive circuit connected to the plurality of coils, the at least one drive circuit and the plurality of coils being arranged to generate a substantially uniform magnetic field in a region within the accommodation cage.

14. The device according to claim 13, wherein the magnetic field in the region within the accommodation cage has an intensity of up to 100 gauss and a frequency between about 0.5 and about 50 Hz.

15. The device according to claim 13, wherein the magnetic field has an intensity of about 4 gauss and frequency of about 10 Hz.

16. The device according to claim 13, wherein the plurality of coils comprise coaxial windings.

17. The device according to claim 13, wherein the plurality of coils comprise a cylindrical winding.

18. The device according to claim 16, wherein the plurality of coils comprise coaxial square windings and each coil is fixed on corner structures.

19. The device according to claim 16, wherein the plurality of coils are configured to slide on longitudinal supports to regulate a distance between the plurality of coils themselves.

20. The device according to claim 17, wherein the plurality of coils are configured to slide on longitudinal supports to regulate a distance between the plurality of coils themselves.

21. The device according to claim 13, wherein the drive circuit includes a signal generator and an amplifier.

22. The device according to claim 21, wherein the signal generator supplies a drive signal to the amplifier, and the amplifier drives the plurality of coils of the device.

23. The device according to claim 22, wherein the amplifier is a multichannel amplifier with a channel for each of the coils.

24. The device according to claim 23, wherein the amplifier is a class D switching amplifier.

25. The device according to claim 22, wherein the signal generator supplies at least one of a sinusoidal, triangular or square wave signal at an input of the amplifier.

26. A method of treating malaria, comprising applying a magnetic field of given intensity and frequency.

27. The method according to claim 26, wherein the magnetic field has an intensity of up to about 100 gauss and a frequency between about 0.5 and about 50 Hz.

28. The method according to claim 26, wherein the magnetic field has an intensity of about 4 gauss and a frequency of about 10 Hz.
